(19) United States
(12) Patent Application Publication
(10) Pub. No.: US 2018/0273463 A1
(43) Pub. Date: Sep. 27, 2018

(54) CATALYST HAVING A MODIFIED SILICON CARBIDE SUPPORT AND ITS USE AS A HYDROGENATION CATALYST

(71) Applicant: UOP LLC, Des Plaines, IL (US)

(72) Inventors: Raelynn M. Miller, LaGrange, IL (US); Alakananda Bhattacharyya, Glen Ellyn, IL (US); David S. Lafyatis, Schaumburg, IL (US); Amanda L. Stolarski, Palos Heights, IL (US); Rodrigo Lobo, Des Plaines, IL (US); Madan Bhasin, South Charleston, WV (US)

(21) Appl. No.: 15/996,394

(22) Filed: Jun. 1, 2018

Related U.S. Application Data
(63) Continuation of application No. PCT/US2016/066586, filed on Dec. 14, 2016.
(60) Provisional application No. 62/269,379, filed on Dec. 18, 2015.

(51) Int. Cl. C07C 51/487; C07C 51/255; C07C 63/26; B01J 23/44; B01J 27/224; B01J 21/06; B01J 35/10

(52) U.S. Cl. CPC C07C 51/487; C07C 51/255; C07C 63/26; B01J 35/1009; B01J 27/224; B01J 21/06; B01J 23/44

(57) ABSTRACT

Impure aromatic carboxylic acids such as are obtained by liquid phase oxidation of feed materials comprising aromatic compounds with substituent groups oxidizable to carboxylic acid groups, or comprising aromatic carboxylic acid and one or more aromatic carbonyl impurities that form hydrogenated species more soluble in aqueous solvents or with less color or color-forming tendencies than the aromatic carbonyl impurity, are purified to an aromatic carboxylic acid product with lower levels of impurities by a process comprising contacting an aqueous solution comprising the impure aromatic carboxylic acid with hydrogen at elevated temperature and pressure with an attrition-resistance, acid stable catalyst composition comprising at least one hydrogenation catalyst metal and a support comprising relatively high surface area of high porosity silicon carbide with low levels of iron and alkali metal impurities. The support may further contain titanium or rare earth metals.

### CROSS-REFERENCE TO RELATED APPLICATIONS

This application is a Continuation of copending International Application No. PCT/US2016/066586 filed Dec. 14, 2016, which application claims priority from U.S. Provisional Application No. 62/269,379 filed Dec. 18, 2015, now expired, the contents of which cited applications are hereby incorporated by reference in their entirety.

### FIELD OF THE INVENTION

This invention relates to treating impure aromatic carboxylic acids to reduce levels of impurities and, more particularly, to purifying an impure terephthalic acid, such as a crude terephthalic acid product made by oxidation of a feed material comprising para-xylene, by catalytic hydrogenation of an aqueous solution of the product to be treated at elevated temperature and pressure in the presence of a catalyst comprising a metal such as palladium having catalytic activity for hydrogenation and a support comprising silicon carbide with a significantly reduced level of impurities and increased porosity.

### BACKGROUND OF THE INVENTION

Purification of aromatic carboxylic acids by catalytic hydrogenation generally involves contacting an aqueous solution comprising an impure-acid product containing a desired aromatic carboxylic acid and impurities, such as a crude product made by oxidation of alkyl or other substituted aromatic feed materials, with hydrogen at elevated temperature and pressure in the presence of a catalyst comprising a metal with catalytic activity for hydrogenation disposed substantially on the surface of a solid carrier that is inert to the reactants and substantially insoluble in the liquid reaction mixture under reaction conditions. Hydrogenation of the aqueous solution of impure product permits separation of a purified, solid product from the hydrogenated reaction solution with a greater part, of impurities that affect quality of the desired aromatic carboxylic acid product contained in the remaining mother liquor as a result of hydrogenation either to species with greater aqueous solubility so that they remain dissolved in the mother liquor or to species less detrimental to quality if present in the purified product.

Terephthalic acid is widely used for the manufacture of polyethylene terephthalate polyesters used to make fibers, films and bottles, among other things, and is commonly made by heavy metal-catalyzed, liquid phase oxidation of para-xylene feed materials. The resulting crude oxidation product typically comprises the desired terephthalic acid and amounts of oxidation intermediates and other by-products, such as 4-carboxybenzaldehyde and p-toluic acid, and colored or color-forming species such as 2,6-dicarboxyfluorenone and 2,6-dicarboxyanthroquinone. Crude product with up to about 5,000 to 10,000 parts per million by weight (“ppmw”) 4-carboxybenzaldehyde is not uncommon, and even amounts as low as 25 ppmw may be or correlate with impurity levels that may be detrimental to color of polyesters. As known from U.S. Pat. No. 3,584,039, purification of such crude and other impure terephthalic acid products by catalytic hydrogenation of an aqueous solution thereof at elevated temperatures and pressures converts 4-carboxybenzaldehyde to hydroxymethyl benzoic acid, which in turn is converted to p-toluic acid, both of which are more soluble in the aqueous reaction liquid than terephthalic acid. Solid terephthalic acid with reduced levels of 4-carboxybenzaldehyde compared to the starting crude product can be crystallized from the reaction liquid while hydroxymethyl benzoic acid and p-toluic acid resulting from hydrogenation of 4-carboxybenzaldehyde remain in solution. Hydrogenation of the crude product also converts colored and color-forming benzil, fluorenone and anthraquinone species such as 2,6-dicarboxyfluorenone and 2,6-dicarboxyanthroquinone, to corresponding colorless or less colored hydrogenated compounds. Related purification of impure isophthalic acid products, commonly made by liquid phase oxidation of meta-xylene feed materials, is disclosed in U.S. Pat. No. 4,933,492.

Conventional catalysts for practical commercial applications of such processes commonly comprise palladium carried on an inert, granular carbon support. Carbon supports are readily obtainable and chemically stable in the high temperature, acidic environments of purification reaction processes. However, carbon supports tend to be fragile and carbon-supported catalysts are easily damaged by process flow, pressure, and temperature upsets. Even minor damage can produce fine catalyst particles that can carry over with the product from a purification reactor and contaminate the purified product. In the case of purified terephthalic acid products, this contamination typically is manifested by high particulate contamination levels as indicated by standard measures such as L* values, which indicate grayness on a scale of 100 (corresponding to white or colorless) to 0 (corresponding to black), with values below 98 generally being considered poor for purified terephthalic acid.

More serious damage to carbon-supported catalysts can degrade a catalyst bed so extensively that reactor pressure drop becomes unacceptable. In such cases, the entire catalyst bed must be replaced. Another consequence of fragility of conventional carbon supports is loss of catalyst metals over the lifetime of a catalyst bed due to fines generated during upsets and catalyst loading and maintenance procedures. Spent catalyst beds containing 70% or even less of their initial catalyst metal contents are not uncommon. Loss of catalyst metals not only diminishes catalyst activity and lifetime but also creates a financial penalty from the lost metals themselves, especially in the case of expensive metals such as palladium.

A stronger catalyst support could reduce these difficulties with carbon supports. In that respect, properties such as crush strength and resistance to abrasion, formation of catalyst fines and loss of catalyst metals under conditions of handling, storage and use are important attributes of a support. Improvements in properties, however, are sometimes difficult to achieve without sacrifices in others. Beyond strength and abrasion resistance, utility of a support with particular catalyst metals for particular chemical reactions on a scale and under conditions suited to practical process applications is impacted, often unpredictably, by its activity, or lack of activity, for side reactions and affinity, or lack thereof, for adsorption and other surface phenomena under conditions of use, surface characteristics, such as surface area, pore size and volume, suited to facile and adequate catalyst metal loadings in catalyst preparation and effective reaction rates during catalyst use, and other factors.

Silicon carbide, as conventionally used as an abrasive and in refractory materials such as firebrick, rods and tubes, is commonly prepared commercially by fusing sand and coke in an electric furnace at temperatures above 2,200° C. The resulting silicon carbide forms extremely hard, dark, iridescent crystals that are free of porosity, insoluble in water and other common solvents and stable at high temperatures. The compositions are said to have utility as supports for catalysts for petrochemical and high temperature reactions, such as rhodium or platinum catalysts for conversion of carbon monoxide and unburned hydrocarbons to CO₂ and nitrogen oxide to NO₂ in catalytic converters for internal combustion-engines, cobalt-molybdenum catalysts for petrochemical hydrotreatments such as hydrodesulphurization and hydrodemetallation, and for controlled oxidations to convert methane and other low molecular weight hydrocarbons to higher hydrocarbons.

While silicon carbides have been reported to be useful as supports for catalysts for hydrogenation of impure aromatic carboxylic acids or for similar reactions such as in U.S. Pat. No. 8,492,583, there remains a need to improve these materials to achieve improved product quality, performance and longer life span that are necessary to be commercially viable.

### SUMMARY OF THE INVENTION

This invention provides a catalytic process for purification of an impure aromatic carboxylic acid product to an aromatic carboxylic acid product containing lower levels of impurities than prior art processes. The process uses a catalyst comprising a silicon carbide support having at least one hydrogenation catalyst metal dispersed homogeneously throughout the support. The silicon carbide of the catalyst has much lower impurity levels than prior art silicon carbide and with improved abrasion and attrition resistance compared to conventional carbon supports for aromatic acid purification catalysts and improved stability in acidic solutions, even at high temperatures. In addition, these catalysts have the hydrogenation catalyst metal dispersed throughout the silicon carbide which further improves attrition resistance. A further difference is that the silicon carbide that is used in the present invention has macrovoids which result in a bulk density much lower than prior art silicon carbide. These features also characterize the catalysts used according to the process. The improved properties of the catalysts, and the supports from which they are prepared, contribute to improvements in catalyst lifetime, process stability and reduced presence of catalyst particles in the purified product according to the current process as compared to processes using conventional carbon-supported catalysts. The process provides product quality comparable to that obtained in hydrogenation processes using catalysts with conventional carbon supports.

In one embodiment, the impure aromatic carboxylic acid product that is purified according to the invented process comprises a crude aromatic carboxylic acid product obtained by liquid phase oxidation of a feed material comprising an aromatic compound with oxidizable substituents. The crude product of such oxidations comprises the aromatic carboxylic acid and one or more oxidation intermediates or by-products. Although the specific chemical compositions of intermediates and by-products will vary somewhat depending on factors such as the composition of the oxidation feed material and oxidation reaction conditions, and even for a given feed material are not fully known, they are known to comprise one or more aromatic carbonyl compounds, such as benzaldehydes, carboxybenzaldehydes, fluorenones and anthraquinones, that cause or correlate with undesirable color of the desired aromatic carboxylic acid product or of polyesters made therefrom and can be hydrogenated to species more soluble in aqueous solution than the aromatic carbonyl compounds and the aromatic carboxylic acid or to species with less color or color-forming tendencies. Hydrogenation according to the invention converts carbonyl substituents on aromatic nuclei of the impurities to corresponding hydrogenated groups, such as hydroxyalkyl and/or alkyl groups but without significant decarboxylation or ring hydrogenation reactions. Accordingly, in addition to impure aromatic carboxylic acid products comprising a crude aromatic carboxylic acid product obtained by liquid phase oxidation of a feed material comprising an aromatic compound with oxidizable substituents, the invention is useful for purification of impure aromatic carboxylic acid products comprising an aromatic carboxylic acid and such aromatic carbonyl impurities, whether present as intermediates or by-products from prior manufacturing steps or from any other source. Thus, in another aspect, the invention provides a process for purifying an impure aromatic carboxylic acid product comprising at least one aromatic carboxylic acid and at least one aromatic carbonyl impurity that forms a hydrogenated carbonyl-substituted aromatic product with greater solubility in aqueous solution or with less color or color-forming tendencies than the aromatic carbonyl impurity.

Briefly, the process according to the invention is a process for purifying an impure aromatic carboxylic acid comprising contacting with hydrogen under hydrogenation reaction conditions and in the presence of a catalyst an aqueous solution comprising impure aromatic carboxylic acid, wherein the catalyst comprises at least one hydrogenation catalyst metal dispersed homogeneously throughout the support comprising silicon carbide having a BET surface area of at least about 1 m²/g, the catalyst has an initial attrition loss according to ASTM D-4058 less than about 1.2 wt % and the silicon carbide is substantially stable in the aqueous solution under the hydrogenation reaction conditions. In embodiments of the invention, the BET surface area may be at least 3 m²/g, at least 5 m²/g or at least 10 m²/g. Aromatic carboxylic acid product with reduced impurities is separated from the hydrogenated reaction solution, leaving impurities and their hydrogenated products substantially in solution in the resulting mother liquor. The amount of impurities can vary but is generally less than 5 wt %, more often less than 1 wt % and most often less than 0.5 wt % or 0.3 wt %. The level of impurities is reduced by at least 75%, at least 90%, at least up to 95% or as high as about 98%.

Another embodiment of the invention provides a process for treating an impure aromatic carboxylic acid product that comprises a crude terephthalic acid obtained by a liquid phase oxidation of a feed material comprising para-xylene to a product that comprises terephthalic acid and at least one oxidation intermediate or by-product comprising forming an aqueous solution comprising the impure aromatic carboxylic acid product and contacting the aqueous solution with hydrogen at a temperature of about 200 to about 325° C. and pressure of about 500 to about 1500 psig in the presence of a catalyst comprising a hydrogenation catalyst metal homogeneously dispersed throughout a support comprising silicon carbide having a BET surface area of at least about 1 m²/g, wherein the catalyst has an initial attrition loss according to ASTM D-4058 of less than about 1.2 wt % and the silicon carbide is substantially stable in the aqueous solution at a temperature of about 200 to about 325° C. and pressure of about 500 to about 1500 psig. In embodiments of the invention, the BET surface area may be at least 3 m²/g, at least 5 m²/g or at least 10 m²/g. In some embodiments of the invention, the support that is used may further contain titanium or a rare earth metal such as lanthanum. The amount of impurities can vary but is generally less than 5 wt %, more often less than 1 wt % and most often less than 0.5 wt % or 0.3 wt %. The level of impurities is reduced by at least 75%, at least 90 %, at least up to 95% or as high as 98%.

In another embodiment the invention provides a process for treating an impure aromatic carboxylic acid product that comprises terephthalic acid and at least one of 4-carboxybenzaldehyde, hydroxymethyl benzoic acid, p-toluic acid, 2,6-dicarboxyfluorenone, 2,6-dicarboxyanthroquinone, 2,4′,5-tricarboxybiphenyl, 2,5-dicarboxyphenyl-4-carboxyphenyl methane, 3,4′- and 4,4′-dicarboxybiphenyl, and 2,6-dicarboxyfluorene comprising forming an aqueous solution comprising the impure aromatic carboxylic acid product and contacting the aqueous solution with hydrogen at a temperature of about 200 to about 325° C. and pressure of about 500 to about 1500 psig in the presence of a catalyst comprising a hydrogenation catalyst metal homogenously dispersed throughout a support comprising silicon carbide having a BET surface area of at least about 1 m²/g, wherein the catalyst has an initial attrition loss according to ASTM D-4058 of less than about 1.2 wt % and the silicon carbide is substantially stable in the aqueous solution at a temperature of about 200 to about 325° C. and a pressure of about 500 to about 1500 psig. In embodiments of the invention, the BET surface area may be at least 3 m²/g, at least 5 m²/g or at least 10 m²/g. In some embodiments of the invention, the support that is used may further contain titanium or a rare earth metal such as lanthanum. The amount of impurities can vary but is generally less than 5 wt %, more often less than 1 wt % and most often less than 0.5 wt % or 0.3 wt %. The level of impurities is reduced by at least 75%, at least 90%, at least up to 95% or as high as 98%.

It was found that improved catalyst performance was the result of two main improvements over the prior art. The silicon carbide support that was used had a significantly lower level of iron and alkali metal impurities with less than 300 ppmw total impurity and preferably less than 100 ppmw or less than 50 ppmw iron and less than 100 ppmw or less than 50 ppmw sodium. In some embodiments of the invention, the support that is used may further contain titanium or a rare earth metal such as lanthanum. In addition, advantages in catalyst performance were found through an increase in the porosity of the silicon carbide support. An increase in porosity in both mesopores and macropores was found to increase the conversion of 4-carboxybenzaldehyde. The resulting catalyst had lower impurities, greater hardness, stability, dispersion of palladium, and re-usability.

### DETAILED DESCRIPTION

Processes according to the invention provide desirable improvements in purity of impure aromatic carboxylic acids, typically with improved catalyst life and reduced reactor plugging and product contamination due to catalyst fines, than with carbon-supported catalysts. Aromatic carboxylic acid purification processes according to the invention may be conducted under more robust conditions conducive to higher throughputs or production rates than with granular carbon-supported catalysts as a consequence of the improved strength and attrition resistance of the silicon carbide supports to the catalysts used according to the invention. Increased lifetime of the silicon carbide-supported catalysts allows longer operating periods between catalyst additions or replacements, and risk of reactor upsets due to plugging with catalyst fines and fugitive catalyst metal particles is reduced. The silicon carbide supports and catalysts based thereon also have good resistance to acidic environments, especially after conditioning in aqueous or aqueous acidic liquids according to an aspect of the invention. Significantly, a catalyst bed comprising solid particles of the silicon carbide-supported hydrogenation metal used according to the invention supported or suspended in an aqueous solution containing up to about 50 wt % aromatic carboxylic acid at temperatures up to about 325° C. and under pressures up to about 1500 psig, is substantially resistant to loss of catalyst fines and catalyst metal for prolonged periods of time, and with insignificant presence of silicon and silicon oxides in the purified product and reaction solution residues. The acid and high temperature resistance of the catalysts make them versatile for use not only in purification of aromatic carboxylic acids but also in other processes operated at high temperatures or involving acidic reactants, solvents, products or by-products. The increased strength and attrition resistance of the silicon carbide-supported hydrogenation catalysts as compared to carbon-supported catalysts, together with their ability to withstand acidic and high temperature conditions in use, afford greater opportunities for recovery of catalyst metals and re-use of supports than do conventional carbon supports.

Aromatic carboxylic acids of the impure product that are treated according to the invented process to reduce levels of impurities generally contain one or more aromatic nuclei and 1 to about 4 carboxylic acid groups. Examples include benzoic acid, phthalic acid, terephthalic acid, isophthalic acid, trimesic acid, trimellitic acid, and naphthalene dicarboxylic acids. Preferred aromatic carboxylic acids are dicarboxylic acids with a single aromatic ring and especially terephthalic acid. In commercial practice, these acids are often obtained by heavy metal-catalyzed, liquid phase oxidation of feed materials comprising aromatic compounds with oxidizable substituents, such as toluene, xylenes, trimethylbenzenes and dimethyl and diethyl naphthalenes.

The impure aromatic carboxylic acid to be purified according to the invention also comprises one or more impurities. In the case of an impure aromatic carboxylic acid comprising a crude product obtained by liquid phase oxidation of feed materials comprising aromatic compounds with oxidizable substituent groups, impurities comprise oxidation by-products or intermediates. In the case of a crude terephthalic acid product obtained by liquid phase oxidation of feed materials such as p-xylene, common oxidation intermediates and by-products are one or more of 4-carboxybenzaldehyde, hydroxymethyl benzoic acid, p-toluic acid, 2,6-dicarboxyfluorenone, 2,6-dicarboxyanthroquinone, 2,4′,5-tricarboxybiphenyl, 2,5-dicarboxyphenyl-4-carboxyphenyl methane, 3,4′- and 4,4′-dicarboxybiphenyl, and 2,6-dicarboxyfluorene. Among known impurities, at least 4-carboxybenzaldehyde, 2,6-dicarboxyfluorenone and 2,6-dicarboxyanthroquinone are known to cause or correlate with color of terephthalic acid or its polyesters.

More generally, and without regard to source or method of manufacture of the impure aromatic carboxylic acid to be purified, impurities that can be hydrogenated according to the invention to purify impure aromatic carboxylic acids in which they are present commonly comprise one or more aromatic carbonyl compounds, such as aromatic aldehydes and ketones with one or more aromatic rings. Specific examples include benzaldehyde, 2-, 3- and 4-carboxybenzaldehydes, 2,6-dicarboxyfluorenone, 2,4′,5-tricarboxybiphenyl, 2,5-dicarboxyphenyl-4-carboxyphenyl methane, 3,4′- and 4,4′-dicarboxybiphenyl and 2,6-dicarboxyanthroquinone. Hydrogenation of such compounds results in conversion of carbonyl groups to corresponding hydroxyalkyl and alkyl groups. The resulting hydrogenated species are typically more soluble in aqueous solvents than the original carbonyl species and more than the desired aromatic acid product, or are less colored or less prone to imparting color to polyesters or other products made from the desired product, thereby facilitating separation of the more soluble hydrogenated carbonyl compounds from the desired product by crystallization and leaving a greater portion of colored or color-forming species in the reaction liquid or mother liquor from which the desired product is crystallized. Hydrogenation according to the invention is selective to the carbonyl species, and proceeds without substantial ring hydrogenation of either the aromatic carbonyls or of the desired aromatic acids, and also without substantial decarbonylation or decarboxylation of carboxylic acid substituents on the aromatic rings.

Amounts of impurities, such as oxidation by-products and intermediates and/or aromatic carbonyl compounds, present in the impure aromatic carboxylic acids to be treated according to the invention vary with the nature and source of the impurities. Generally, any amount of such impurities may be present without hindering effectiveness of the invention, although if present at high enough levels, other separation techniques may be more practical or economically efficient. Aromatic carboxylic acids as obtained in liquid phase oxidations of alkyl aromatic feed materials often contain as much as 1 to 2 wt % impurities, with up to about 1 wt % being more common in commercial practice. Most often the amount of these impurities is in a range up to about 0.5 wt % in some cases and in other cases up to 0.3 wt %. The level of impurities needs to be significantly reduced. It may be reduced by at least 75%, at least 9%, at least up to 95% or as high as 98%.

Hydrogenation of impure aromatic carboxylic acids to reduce impurities levels according to the invention is conducted with the impure acid in aqueous solution. Water is a preferred solvent for the process although lower monocarboxylic acids, alone or mixed with water, may also be used. When using water as the purification solvent, minor amounts of acetic acid, which is a common solvent used in manufacture of crude aromatic carboxylic acids, may be present as a result of incomplete removal thereof from the product to be purified or other sources. Concentrations of impure aromatic carboxylic acid to be treated in the purification solvent generally are low enough that the impure acid is substantially dissolved and high enough for practical process operations and efficient use and handling of solvents. Suitably, solutions comprising about 5 to about 50 parts by weight impure aromatic carboxylic acid per hundred parts by weight solution at process temperatures provide adequate solubility for practical operations. Preferred feed solutions contain about 10 to about 40 wt % and more preferably about 20 to about 35 wt % impure aromatic carboxylic acid at the temperatures used for treatment.

Purification of the aqueous solution is conducted at elevated temperatures and pressures. Temperatures range from about 200 to about 370° C., with about 225 to about 325° C. being preferred and about 240 to about 300° C. being most preferred. Purification is conducted at a pressure sufficient to maintain a liquid phase comprising the aqueous reaction solution. Total pressure is at least equal to, and preferably exceeds, the sum of the partial pressures of the hydrogen gas introduced to the process and water vapor that boils off from the aqueous reaction solution at the temperature of operation. Preferred pressures are about 500, and more preferably about 1000, to about 1500 psig.

The aqueous solution of impure aromatic carboxylic acid is contacted with hydrogen under hydrogenation conditions as described above in a suitable reaction vessel capable of withstanding the temperature and pressures under which hydrogenation is conducted and also the acidic nature of the liquid reaction mixture. A preferred reactor configuration is a cylindrical reactor with a substantially central axis positioned with the axis vertically disposed when the reactor is in use. Both upflow and downflow reactors can be used. Catalyst typically is present in the reactor in one or more fixed beds of particles maintained with a mechanical support for holding the catalyst particles in the bed while allowing relatively free passage of reaction solution therethrough. A single catalyst bed is often preferred although multiple beds of the same or different catalyst or a single bed layered with different catalysts, for example, with respect to particle size, hydrogenation catalyst metals or metal loadings, or with catalyst and other materials such as abrasives to protect the catalyst, also can be used and may provide benefits. Mechanical supports in the form of flat mesh screens or a grid formed from appropriately spaced parallel wires are commonly employed. Other suitable catalyst retaining means include, for example, a tubular Johnson screen or a perforated plate. The mechanical support for the catalyst bed is constructed of a material that is suitably resistant to corrosion, due to contact with the acidic reaction solution, and strong enough to efficiently retain the catalyst bed. Most suitably, supports for catalyst beds have openings of about 1 mm or less and are constructed of metals such as stainless steel, titanium or Hastelloy C.

In the process of the invention, aqueous solution of impure aromatic carboxylic acid to be purified is added to the reactor vessel at elevated temperature and pressure at a position at or near the top portion of the reactor vessel, and the solution flows downwardly through the catalyst bed contained in the reactor vessel in the presence of hydrogen gas, wherein impurities are reduced with hydrogen, in many cases forming hydrogenated products with greater solubility in the reaction mixture than the desired aromatic carboxylic acid or with less color or color-forming tendencies. In such a preferred mode, the impure carboxylic acid is purified and the purified product is removed from the reactor vessel at a position at or near a lower portion or the bottom of the reactor.

The reactor may be operated in several modes. In one operating mode, a predetermined liquid level may be maintained in the reactor and, for a given reactor pressure, hydrogen can be fed at a rate sufficient to maintain the predetermined liquid level. The difference between the actual reactor pressure and the vapor pressure of the vaporized purification solution present in the reactor head space is the hydrogen partial pressure in the head space. Alternatively, hydrogen can be fed mixed with an inert gas such as nitrogen or water vapor, in which case the difference between the actual reactor pressure and the vapor pressure of the vaporized reaction solution present is the combined partial pressure of hydrogen and the inert gas admixed therewith. In this case the hydrogen partial pressure may be calculated from the known relative amounts of hydrogen and inert gas present in the admixture.

In another operating mode, the reactor can be filled with the aqueous liquid reaction mixture so that there is no reactor vapor space. In such an embodiment, the reactor is operated as a hydraulically full system with dissolved hydrogen being fed to the reactor by flow control. In such an embodiment, the concentration of hydrogen in solution may be modulated by adjusting the hydrogen flow rate to the reactor. If desired, a pseudo-hydrogen partial pressure value may be calculated from the solution hydrogen concentration which, in turn, may be correlated with the hydrogen flow rate to the reactor.

When operating such that process control is effected by adjusting the hydrogen partial pressure, the hydrogen partial pressure in the reactor is preferably in the range of 10 pounds per square inch gauge to 200 pounds per square inch gauge (69-1379 kPa) or higher, depending on pressure rating of the reactor, impurities levels of the impure aromatic carboxylic acid, activity and age of the catalyst and other considerations known to persons skilled in the art. In the operating mode in which process control is effected by directly adjusting the hydrogen concentration in the feed solution, the latter usually is less than saturated with respect to hydrogen and the reactor itself is hydraulically full. Thus, an adjustment of the hydrogen flow rate to the reactor will result in the desired control of hydrogen concentration in the solution.

After hydrogenation, the hydrogenated stream comprising aromatic carboxylic acid and hydrogenated aromatic impurities having greater solubility in the aqueous reaction liquid than their unhydrogenated precursors is cooled to separate a purified, solid aromatic carboxylic acid from the hydrogenated reaction liquid, leaving a liquid product, frequently referred to as a purification mother liquor, in which hydrogenated impurities remain dissolved. Separation is commonly achieved by cooling to a crystallization temperature, which is sufficiently low for crystallization of the purified aromatic acid to occur, thereby producing crystals within the liquid phase. The crystallization temperature is sufficiently high so that impurities and their reduction products resulting from hydrogenation remain dissolved in the liquid phase. Crystallization temperatures generally range up to 160° C. and preferably up to about 150° C. In continuous operations, separation normally comprises removing the hydrogenated reaction solution from the purification reactor and crystallization of aromatic carboxylic acid in one or more crystallization vessels. When conducted in a series of stages or separate crystallization vessels, temperatures in the different stages or vessels can be the same or different and preferably decrease from each stage or vessel to the next. Thereafter, crystallized, purified aromatic carboxylic acid product is recovered from the mother liquor, while the hydrogenated impurities remain dissolved in the mother liquor. Recovery of the crystallized purified product is commonly conducted by centrifuging or by filtration. Physical integrity and chemical stability of the catalysts used according to the invention are such that silicon/silica content of the purified aromatic carboxylic acid products obtained from the invented process typically is less than about 15 ppmw, and preferably less than about 10 ppmw. Silicon content of purification mother liquor remaining, after separation of purified aromatic carboxylic acid product from the hydrogenated reaction solution is less than about 500 ppmw and preferably less than about 100 ppmw.

Purification reactor and catalyst bed configurations and operating details and crystallization and product recovery techniques and equipment useful in the process according to this invention are described in further detail in U.S. Pat. No. 4,629,715, U.S. Pat. No. 4,892,972, U.S. Pat. No. 5,175,355, U.S. Pat. No. 5,354,898, U.S. Pat. No. 5,362,908 and U.S. Pat. No. 5,616,792.

The catalyst used in invented process comprises a relatively high surface area support comprising silicon carbide and one or more metals having catalytic activity for hydrogenation of impurities in impure aromatic carboxylic acid products, such as oxidation intermediates and by-products and/or aromatic carbonyl species. Suitable catalyst metals are the Group VIII metals of the Periodic Table of Elements (IUPAC version), including palladium, platinum, rhodium, osmium, ruthenium, iridium, and combinations thereof. Palladium or combinations of such metals that include palladium are most preferred. Suitable metal loadings generally are about 0.1 wt % to about 5 wt % based on total weight of the support and catalyst metal or metals. Preferred catalysts for conversion of impurities present in impure aromatic carboxylic acid products comprising crude terephthalic acid obtained by liquid phase oxidation of a feed material comprising para-xylene contain about 0.1 to about 3 wt % and more preferably about 0.2 to about 1 wt % hydrogenation metal. For such uses, the metal most preferably comprises palladium.

For practical applications, the catalyst is most preferably used in particulate form, for example as pellets, extrudate, spheres or granules, although other solid forms also are suitable. Particle size of the catalyst is selected such that a bed of catalyst particles is easily maintained in a suitable reactor for the purification process but permits flow of the purification reaction mixture through the bed without undesirable pressure drop. Preferred average particle sizes are such that catalyst particles pass through a 2-mesh screen but are retained on a 24-mesh screen (U.S. Sieve Series) and more preferably pass through a 4-mesh screen but are retained on a 12-mesh and, most preferably, on an 8-mesh screen.

The catalyst used in the process has BET surface areas of at least about 1 m2/gram. While low by comparison to surface areas of conventional carbon-supported catalysts, surface areas are generally an order of magnitude greater than those of conventional silicon carbides used as abrasives and are satisfactory for use according to the invented process. In embodiments of the invention, the BET surface area may be at least 3 m²/g, at least 5 m²/g or at least 10 m²/g. Preferably, surface areas of the catalysts are at least about 15, and more preferably at least about 20 m²/gram. Catalyst surface area is substantially attributable to surface area of the support and, while known high surface area silicon carbides are generally believed to have surface areas in the range of 10 to about 200 m²/gram, still higher surface area silicon carbides and catalysts comprising such supports and one or more hydrogenation catalyst metals as described herein are contemplated according to the invention provided they are resistant to attrition and stable in aqueous acid solutions at elevated temperatures as described herein.

Attrition resistance of the catalysts used in the invented process is determined according to ASTM D-4058, with attrition loss of the catalysts before use in the invented process, also referred to herein as initial attrition loss, being less than about 1.2 wt % and preferably less than about 1 wt %. Attrition resistance of the catalysts is attributable to that of the silicon carbide support included in the catalyst and the dispersion of the metal, such as palladium throughout the support. The attrition resistance of the relatively high surface silicon carbides used as supports according to the invention is significantly greater than that of conventional carbons used as supports for catalysts for purification of aromatic carboxylic acids.

The catalysts used in the invented process exhibit surprising stability in aqueous acidic solutions, even at the elevated temperatures and pressures used in the invented process. In general, the catalysts lose less than about 2% of their weight, and preferably no more than about 1 wt %, after 20 days exposure to 20 wt % solution of terephthalic acid at about 275° C. and 850 psig.

The silicon carbide support used to prepare the catalysts used in the invented process generally have BET surface areas and attrition resistance according to ASTM D-4058 as described above with regard to the catalysts themselves. The silicon carbide has low iron content with iron limited to 100 ppmw and preferably less than 50 ppmw which is lower than prior art materials that contain much higher levels of iron such as about 500 to 600 ppmw iron as well as much lower levels of alkali metals such as sodium which is also less than 100 ppmw and preferably less than 50 ppmw. The acid stability of the supports is greater than that of the silicon carbides described in U.S. Pat. No. 3,584,039. Supports can be obtained by any suitable technique for making relatively high surface area, attrition resistant silicon carbides, such as by high temperature reaction of a silicon compound that volatilizes at the reaction temperature with a high surface area carbon. Examples of methods for preparing high surface area, attrition-resistant silicon carbides are found in U.S. Pat. No. 4,914,070, U.S. Pat. No. 5,427,761 and other patents cited herein. In one embodiment of the invention, silicon carbide comprising beta crystallites, and preferably with a substantial absence of alpha crystallites, is a preferred form of silicon carbide. U.S. Pat. No. 6,184,178 discloses preparation of a silicon carbide in beta crystallite form. In some embodiments, the silicon carbide support may also contain titanium or a rare earth metal such as lanthanum. The amount of titanium may vary from 0.01 to 0.05 molar ratio to the silicon plus carbon, but more typically the ratio of Ti/(Si+C) is about 0.020 to 0.04.

A specific example of the high surface area silicon carbide support useful for making the catalysts used according to the invented process is a high purity silicon carbide that is commercially available from SICAT Corporation in the form of 3 mm diameter extrudate and having a BET surface area of 21 m²/gram and attrition loss according to ASTM D-4058 of about ½ to about 1 wt %.

### EXAMPLE 1

The high purity silicon carbide support, provided by SiCAT, was tested in a modified Humble Abrasion test in addition to the Calgon DG-13 Activated Carbon. The 3 mm dia SiCAT support with L/D=1 showed 99.9% recovery of material. In contrast, the 6×12 mesh carbon support showed 98.6% recovery.

### EXAMPLE 2

Palladium nitrate salt was used to impregnate the high purity silicon carbide support via incipient wetness technique followed by a slow moisture controlled drying step to reduce the level of palladium wicking outward while drying. Testing of this finished (oxidized and reduced) catalyst in an autoclave reactor at 240° C. with excess H2 and 7400 wppm 4-carboxybenzaldehyde in terephthalic acid showed 94.3% 4-carboxybenzaldehyde conversion, somewhat lower than the in-house Pd/carbon catalyst prepared by the same methodology with conversion at 99.8 wt %. However, the Pd/carbon catalyst showed a palladium loss of 27.3 wt % whereas the Pd/SiC catalyst essentially showed no palladium loss after testing.

### EXAMPLE 3

This same finished (oxidized and reduced) catalyst shows 20.9% palladium dispersion and an average palladium crystallite size of 54 Å. After testing, the palladium dispersion of the Pd/SiC catalyst decreased to only 14.6% with an average palladium crystallite size of 77 Å. Further, SEM backscatter imaging shows the palladium to be maintained throughout the extrudate with some migration to the pill's edge after testing.

### EXAMPLE 4

Aging these same catalysts for 10 days under simulated process conditions of 20 wt % terephthalic acid in H₂O, 250° C., and excess H₂O showed only 14.0 wt % palladium loss and palladium dispersion at 14.2% with palladium crystallites at 79 Å indicating this catalyst is more robust than the Pd/Carbon catalyst tested which showed 31.8 wt % palladium loss and palladium crystallites greater than 3000 Å.

### EXAMPLE 5

Testing of aged catalyst described in Example 4 in an autoclave reactor at 240° C. with excess H2 and 7400 wppm 4-carboxybenzaldehyde in terephthalic acid showed that the aged Pd/SiC sample exhibited a 4-carboxybenzaldhyde conversion of 95.1 wt %, statistically showing no loss of activity relative to fresh.

### EXAMPLE 6

Surface modification of the high purity silicon carbide support was performed using 10 wt % nitric acid. Finished catalysts were prepared using the as-received support and the surface modified support by impregnating palladium nitrate via incipient wetness followed by a standard muffle dry, oxidization and reduction. Samples from this set showed 8.8% palladium dispersion on the as received supported catalyst and an increase to 11.7% palladium dispersion on the surface modified catalyst indicating that the acid modification is aiding in creating anchoring sites for the palladium.

### EXAMPLE 7

The Pd/SiC catalyst prepared on the surface modified support shown in Example 6 was aged in a static Parr reactor with 20 wt % acetic acid. The aged catalysts showed minor decrease in palladium dispersion to only 7.4% with 151 Å palladium crystallites.

### EXAMPLE 8

Palladium catalysts prepared on high purity TiC-SiC support, provided by SiCAT, were aged in a static Parr reactor with 20 wt % acetic acid at 200° C. for 4 days. There was no loss of palladium, and further, palladium dispersion data showed no change from fresh. Additionally, imaging shows the palladium to be maintained throughout the pill.

In another embodiment of the invention, it was found that when the silicon carbide support has a higher porosity in the mesoporous and macroporous ranges, there is a higher dispersion of the palladium and increased catalytic activity. Several experiments were performed. Support A shows the lowest Total Intrusion Volume (TIV) at 0.455 ml/g and visually by SEM images no large voids are present, only smaller voids <25 um. Support B shows a greater than five-times increase in porosity in the >3300 Å range and an overall increase in TIV by 40% to 0.637 ml/g. Testing of the Pd/SiC finished catalyst for the purification of terephthalic acid in an autoclave shows conversion of 4-carboxybenzaldehyde increased from 66.1% to 87.6% with the increased macroporosity. Next, support C showed additional increase in porosity in the 1000-3300 Å range from 0.130 ml/g to 0.216 ml/g, and an overall increase in TIV to 0.685 ml/g. Testing of the finished catalyst shows an additional increase in 4-carboxybenzaldehyde conversion to 97.0%. Catalyst preparation methodology was consistent for all samples. Palladium nitrate salt was used to impregnate the acid-modified silicon carbide support via incipient wetness technique followed by a slow moisture controlled drying step to reduce the level of palladium wicking outward while drying.

The catalyst used in the invented process can be made by any suitable method for depositing catalyst metal dispersed homogeneously throughout the support. Typically, support particles, such as pellets, granules, extrudate, are contacted with a solution of catalyst metal or a compound thereof in water or another solvent that is inert to the support and easily removed, after which the solvent is removed, such as by drying at ambient or elevated temperature. Incipient wetness techniques, in which a support is contacted with a solution of the catalyst metal compound in an amount that just wets the support and then the resulting wetted support is dried, are known and well suited to manufacture of the catalysts. Other techniques, such as spraying a solution of catalyst metal compound onto the silicon carbide support also are suitable. Suitable catalyst metal compounds are well known and include nitrates and chlorides, specific examples being palladium chloride and palladium nitrate, both of which are water-soluble. Post-treatments, such as high temperature calcinations in the presence of air or nitrogen, and reduction with hydrogen also can be used if desired and may yield catalysts with additional advantages or characteristics of interest. Pretreatment of the silicon carbide particles used for catalyst preparation by contacting the same with water or an aqueous acid solution at temperatures and pressures sufficient to boil the aqueous liquid while maintaining a liquid phase is beneficial for improving aqueous acid stability of the silicon carbide and catalysts prepared therefrom under hydrogenation conditions used according to the invented process. In the prior art the silicon carbide particles would be treated with an aqueous solution comprising up to about 50 wt % organic carboxylic acid at temperatures in the range of about 100 to about 325° C. and pressures of about 1 to about 100 atmospheres. However, in the present invention such treatment is found to be unnecessary.

In a more specific embodiment of the invention, the impure aromatic carboxylic acid product to be purified according to the invention comprises a crude aromatic carboxylic acid product obtained by liquid phase oxidation of a feed material comprising at least one aromatic compound with substituents oxidizable to a carboxylic acid groups. Such oxidations are commonly conducted in a liquid phase reaction mixture comprising a monocarboxylic acid solvent and water with oxygen in the presence of a heavy metal catalyst.

Feed materials for manufacture of such crude aromatic acid products generally comprise an aromatic hydrocarbon substituted with at least one group that is oxidizable to a carboxylic acid group. The oxidizable substituent or substituents can be an alkyl group, such as a methyl, ethyl or isopropyl group. The substituents also can include one or more groups already containing oxygen, such as a hydroxyalkyl, formyl or keto group. The substituents can be the same or different. The aromatic portion of feedstock compounds can be a benzene nucleus or it can be bi- or polycyclic, such as a naphthalene nucleus. The number of oxidizable substituents on the aromatic portion of the feedstock compound can be equal to the number of sites available on the aromatic portion, but is generally fewer than all such sites, preferably 1 to about 4 and more preferably 1 to 3. Examples of useful feed compounds include toluene, ethylbenzene, o-xylene, p-xylene, m-xylene, 1-formyl-2,4-methylbenzene, 1-hydroxymethyl-4-methylbenzene, 1,2,4-trimethyl-benzene, 1-formyl-2,4-dimethylbenzene, 1,2,4,5-tetramethylbenzene, and alkyl-, acyl-, formyl- and hydroxymethyl-substituted naphthalene compounds, such as 2,6- and 2,7-dimethylnaphthalenes, 2-acyl-6-methylnaphthalene, 2,6-diethylnaphthalene, 2-formyl-6-methylnaphthalene and 2-methyl-6-ethylnaphthalene.

For manufacture of a crude aromatic acid product by oxidation of corresponding aromatic feed pre-cursors, e.g., manufacture of isophthalic acid from meta-disubstituted benzenes, terephthalic acid from para-disubstituted benzenes, trimellitic acid from 1,2,4-trisubstituted benzenes, naphthalene dicarboxylic acids from disubstituted naphthalenes, it is preferred to use relatively pure feed materials, and more preferably, feed materials in which content of the pre-cursor corresponding to the desired acid is at least about 95 wt %, and more preferably at least 98% or even higher. A preferred aromatic feed for use to manufacture terephthalic acid comprises para-xylene. A preferred feed for isophthalic acid comprises meta-xylene. A preferred feed for trimellitic acid comprises 1,2,4-trimethylbenzene.

Oxidant gas used for the liquid phase oxidations comprises molecular oxygen. Air is conveniently used as a source of molecular oxygen. Oxygen-enriched air, pure oxygen and other gaseous mixtures comprising at least about 10% molecular oxygen also are useful.

Catalysts used in such liquid phase oxidations comprise materials that are effective to catalyze oxidation of the aromatic hydrocarbon feed to aromatic carboxylic acid. Preferably, the catalyst is soluble in the liquid oxidation reaction body to promote contact among catalyst, oxygen and liquid feed; however, heterogeneous catalyst or catalyst components may also be used. Typically, the catalyst comprises at least one heavy metal component such as a metal with atomic weight in the range of about 23 to about 178. Examples include cobalt, manganese, vanadium, molybdenum, chromium, iron, nickel, zirconium, cerium or a lanthanide metal such as hafnium. Preferably, catalyst comprising one or both of cobalt and manganese is used. Soluble forms of these metals include bromides, alkanoates and bromoalkanoates; specific examples include cobalt acetate and bromide, zirconium acetate and manganese acetate and bromide.

The catalyst preferably is used in combination with a promoter. The promoter is used to promote oxidation activity of the catalyst metal, preferably without generation of undesirable types or levels of by-products, and is preferably used in a form that is soluble in the liquid reaction mixture. Preferably the promoter comprises bromine, including elemental, ionic or organic forms thereof. Examples include Br, HBr, NaBr, KBr, NH.sub.4Br, bromobenzenes, benzyl-bromide, bromo acetic acid, dibromo acetic acid, tetrabromoethane, ethylene dibromide and bromoacetyl bromide. Other promoters include aldehydes and ketones, such as acetaldehyde and methyl ethyl ketone.

A solvent for the feed material, soluble catalyst materials and promoter is desirably used in the process. Solvents comprising an aqueous carboxylic acid, and especially a lower alkyl (e.g., C₁ to C₆) monocarboxylic acid, are preferred because they tend to be only sparingly prone to oxidation under typical oxidation reaction conditions used for manufacture of aromatic acids, and can enhance catalytic effects in the oxidation. Specific examples of suitable carboxylic acids include acetic acid, propionic acid, butyric acid, benzoic acid and mixtures thereof. Ethanol and other co-solvent materials which oxidize to monocarboxylic acids under the aromatic acid oxidation reaction conditions also can be used as is or in combination with carboxylic acids with good results.

Proportions of the feed, catalyst, oxygen and solvent are not critical and vary not only with choice of feed materials and intended product but also choice of process equipment and operating factors. Solvent to feed weight ratios suitably range from about 1:1 to about 10:1. Oxygen typically is used in at least a stoichiometric amount based on feed but not so great that unreacted oxygen escaping from the liquid body to the overhead gas phase forms a flammable mixture with other components of the gas phase. Catalysts suitably are used in weights providing about 100 to about 3000 ppm catalyst metal based on feed weight. Promoter concentrations also generally range from about 100 to about 3000 ppm based on weight of the liquid feed, with about 0.1 to about 2 milligram-atoms of promoter suitably used per milligram-atom of catalyst metal.

Oxidation of aromatic feed materials to crude product comprising aromatic acid is conducted under oxidation reaction conditions. Temperatures in the range of about 120 to about 250° C. are generally suitable, with about 150 to about 230° C. preferred. Pressure in the reaction vessel is at least high enough to maintain a substantial liquid phase comprising feed and solvent in the vessel. Generally, pressures of about 70-500 psi are suitable, with preferred pressures for particular processes varying with feed and solvent compositions, temperatures and other factors. Solvent residence times in the reaction vessel can be varied as appropriate for given throughputs and conditions, with about 20 to about 150 minutes being generally suited to a range of processes. For processes in which the aromatic acid product is substantially soluble in the reaction solvent, such as in the manufacture of trimellitic acid by oxidation of psuedocumene in acetic acid solvent, solid concentrations in the liquid body are negligible. In other processes, such as oxidation of xylenes to isophthalic or terephthalic acids, solids contents can be as high as about 50 wt % of the liquid reaction body, with levels of about 10 to about 35 wt % being more typical. As will be appreciated by those skilled in the manufacture of aromatic acids, preferred conditions and operating parameters vary with different products and processes and can vary within or even beyond the ranges specified above.

Crude aromatic carboxylic acid products of such liquid phase oxidation processes include impurities comprising oxidation intermediates and by-products, typically including one or more aromatic carbonyl species that cause or correlate with color in the desired aromatic acid product or in polyesters made therefrom. Examples of those intermediates and by-products include aldehydes and ketones such as the carboxybenzaldehydes, fluorenones and dicarboxyanthroquinones described above. Impurities levels up to 2 wt % or even higher, depending on feed materials, operating parameters and process efficiency, are not uncommon and can be enough to affect product quality of the desired carboxylic acid product or downstream products thereof.

In a particular embodiment, the invention is used for the manufacture of a purified aromatic carboxylic acid comprising terephthalic acid from a crude aromatic carboxylic acid product comprising terephthalic acid and impurities obtained by boiling liquid phase oxidation of an aromatic hydrocarbon feed comprising para-xylene. Acetic acid or aqueous acetic acid is a preferred solvent, with a solvent to feed ratio of about 2:1 to about 5:1 being preferred. The catalyst preferably comprises cobalt, manganese or a combination thereof, and a source of bromine soluble in the solvent is preferably used as promoter. Cobalt and manganese preferably are used in amounts providing about 100 to about 800 ppmw based on feed weight. Bromine preferably is present in an amount such that the atom ratio of bromine to catalyst metal is about 0.1:1 to about 1.5:1.

Oxygen-containing gas is provided to the liquid phase reaction mixture at a rate effective to provide at least about 3 moles molecular oxygen per mole of aromatic feed material and, in conjunction with removal of reactor off-gases, such that unreacted oxygen in the vapor space above the liquid reaction body is below the flammable limit. When air is the source of oxygen, the limit is about 8 mole % when measured after removal of condensable compounds.

Oxidation preferably is conducted at temperatures of about 160 to about 225° C. under pressure of about 70-50 psi. Under such conditions, contact of the oxygen and feed material in the liquid body results in formation of solid terephthalic acid crystals, typically in finely divided form. Solids content of the boiling liquid slurry typically ranges up to about 40 wt % and preferably from about 20 to about 35 wt %, and water content typically is about 5 to about 20 wt % based on solvent weight. Boiling of the liquid body for control of the reaction exotherm causes volatilizable components of the liquid body, including solvent and water of reaction, to vaporize within the liquid. Unreacted oxygen and vaporized liquid components escape from the liquid into the reactor space above the liquid. Other species, for example nitrogen and other inert gases that are present if air is used as an oxygen source, carbon oxides, and vaporized by-products, e.g., methyl acetate and methyl bromide, also may be present in the overhead vapor.

Crude product from the oxidation is separated from the liquid reaction mixture, typically by crystallization at reduced temperature and pressure, and the resulting solid is recovered by filtration or centrifuging. The recovered crude terephthalic acid comprises 4-carboxybenzaldehyde, typically in amounts ranging from about 500 to about 5000 ppmw, and frequently up to several hundred ppmw of color formers such as 2,6-dicarboxyfluorenone and 2,6-dicarboxyanthroquinone. Purification of the crude product according to the invention typically reduces levels of 4-carboxybenzaldehyde in the purified terephthalic acid to below about 100 ppmw, preferably about 25 ppmw or less, and color former concentrations to negligible amounts.

### Specific Embodiments

While the following is described in conjunction with specific embodiments, it will be understood that this description is intended to illustrate and not limit the scope of the preceding description and the appended claims.

A first embodiment of the invention is a process for purifying an aromatic carboxylic acid by hydrogenating 4-carboxybenzaldehyde comprising contacting a crude aromatic carboxylic acid feed containing about 0.1 to 1.0 wt % of an aromatic carbonyl compound with hydrogen under hydrogenation conditions at a temperature of about 200 to about 370° C. and in the presence of a catalyst, wherein the catalyst comprises particulates comprising a Group VIII hydrogenation catalyst metal dispersed in a support comprising silicon carbide having a catalyst impurity level less than 300 wt ppm and then recovering a purified product that contains 95% less 4-carboxybenzaldehyde than the crude aromatic carboxylic acid feed. An embodiment of the invention is one, any or all of prior embodiments in this paragraph up through the first embodiment in this paragraph wherein the feed contains about 0.3 to 0.5 wt % 4-carboxybenzaldehyde An embodiment of the invention is one, any or all of prior embodiments in this paragraph up through the first embodiment in this paragraph wherein the catalyst impurities comprise iron and alkali metals. An embodiment of the invention is one, any or all of prior embodiments in this paragraph up through the first embodiment in this paragraph wherein the alkali metal is sodium and wherein the sodium is present at less than about 100 ppm. An embodiment of the invention is one, any or all of prior embodiments in this paragraph up through the first embodiment in this paragraph wherein the iron impurity is present at less than about 50 ppm. An embodiment of the invention is one, any or all of prior embodiments in this paragraph up through the first embodiment in this paragraph wherein the catalyst contains about 0.1 to about 5 wt % hydrogenation catalyst metal. An embodiment of the invention is one, any or all of prior embodiments in this paragraph up through the first embodiment in this paragraph wherein the hydrogenation catalyst metal comprises palladium. An embodiment of the invention is one, any or all of prior embodiments in this paragraph up through the first embodiment in this paragraph wherein the aromatic carboxylic acid comprises terephthalic acid. An embodiment of the invention is one, any or all of prior embodiments in this paragraph up through the first embodiment in this paragraph wherein the aromatic carboxylic acid comprises a crude aromatic carboxylic acid product obtained by liquid phase oxidation of a feed material comprising an aromatic compound with one or more substituents oxidizable to a carboxylic acid group and comprises aromatic carboxylic acid and at least one oxidation intermediate or by-product. An embodiment of the invention is one, any or all of prior embodiments in this paragraph up through the first embodiment in this paragraph wherein the crude aromatic carboxylic acid product comprises terephthalic acid. An embodiment of the invention is one, any or all of prior embodiments in this paragraph up through the first embodiment in this paragraph wherein the aromatic carbonyl compound comprises at least one of benzaldehyde, 2-, 3- and 4-carboxybenzaldehyde, 2,6-dicarboxyfluorenone and 2,6-dicarboxyanthroquinone.

A second embodiment of the invention is a method of manufacturing a purified aromatic carboxylic acid product comprising contacting a feed material comprising an aromatic compound with about 0.3-0.5 wt % oxidizable substituents with oxygen in the presence of a heavy metal catalyst in a liquid reaction mixture under oxidation reaction conditions; separating from the liquid reaction mixture a crude product comprising aromatic carboxylic acid and at least one oxidation intermediate or by-product; forming an aqueous solution comprising the crude product; contacting the aqueous solution with hydrogen in the presence of a catalyst in particulate form under hydrogenation reaction conditions at a temperature of about 200° to about 370° C., wherein the catalyst particles comprise a Group VIII hydrogenation catalyst metal dispersed homogeneously throughout the support comprising silicon carbide having a BET surface area of at least about 1 m²/g wherein the catalyst comprises less than about 100 ppmw iron and a total impurity level of less than 300 ppmw. An embodiment of the invention is one, any or all of prior embodiments in this paragraph up through the second embodiment in this paragraph wherein the support further comprises titanium or a rare earth metal. An embodiment of the invention is one, any or all of prior embodiments in this paragraph up through the second embodiment in this paragraph wherein the catalyst comprises less than about 50 ppmw iron. An embodiment of the invention is one, any or all of prior embodiments in this paragraph up through the second embodiment in this paragraph wherein the oxidation intermediate or by-product comprises at least one carboxybenzaldehyde. An embodiment of the invention is one, any or all of prior embodiments in this paragraph up through the second embodiment in this paragraph wherein the oxidation intermediate or by-product comprises 4-carboxybenzaldehyde. An embodiment of the invention is one, any or all of prior embodiments in this paragraph up through the second embodiment in this paragraph wherein the catalyst metal comprises palladium.

A third embodiment of the invention is a process for purification of an aromatic carboxylic acid product comprising terephthalic acid and about 0.3 to 0.5 wt % of at least one impurity comprising 4-carboxybenzaldehyde, hydroxymethyl benzoic acid, p-toluic acid, 2,6-dicarboxyfluorenone, 2,6-dicarboxyanthroquinone, 2,4′,5-tricarboxybiphenyl, 2,5-dicarboxyphenyl-4-carboxyphenyl methane, 3,4′- and 4,4′-dicarboxybiphenyl, and 2,6-dicarboxyfluorene comprising contacting an aqueous solution of the product with hydrogen in the presence of a catalyst in particulate form under hydrogenation reaction conditions at a temperature of about 200° to about 370° C., wherein the catalyst particles comprises a Group VIII hydrogenation catalyst metal dispersed homogeneously throughout the support comprising silicon carbide having a BET surface area of at least about 1 m²/g, the catalyst has an initial attrition loss according to ASTM D-4058 up to about 1.2 wt % and the silicon carbide comprises less than about 200 ppm impurities wherein at least 97 wt % of the impurity comprising 4-carboxybenzaldehyde, hydroxymethyl benzoic acid, p-toluic acid, 2,6-dicarboxyfluorenone, 2,6-dicarboxyanthroquinone, 2,4′,5-tricarboxybiphenyl, 2,5-dicarboxyphenyl-4-carboxyphenyl methane, 3,4′- and 4,4′-dicarboxybiphenyl, and 2,6-dicarboxyfluorene is removed. An embodiment of the invention is one, any or all of prior embodiments in this paragraph up through the third embodiment in this paragraph wherein the impurities comprise iron and alkali metals. An embodiment of the invention is one, any or all of prior embodiments in this paragraph up through the third embodiment in this paragraph wherein the catalyst comprises less than about 100 ppm iron and 100 ppm sodium.

The invention is further described in the following examples, which are presented for purposes of illustration, not limitation.

1. A process for purifying an aromatic carboxylic acid by hydrogenating 4-carboxybenzaldehyde comprising contacting a crude aromatic carboxylic acid feed containing about 0.1 to 1.0 wt % of an aromatic carbonyl compound with hydrogen under hydrogenation conditions at a temperature of about 200° to about 370° C. and in the presence of a catalyst, wherein the catalyst comprises particulates comprising a Group VIII hydrogenation catalyst metal dispersed homogeneously in a support comprising silicon carbide having a catalyst impurity level less than 300 wt ppm and then recovering a purified product that contains 95% less 4-carboxybenzaldehyde than said crude aromatic carboxylic acid feed.

2. The process of claim 1 wherein said feed contains about 0.3 to 0.5 wt % 4-carboxybenzaldehyde.

3. The process of claim 1 wherein said catalyst impurities comprise iron and alkali metals.

4. The process of claim 3 wherein said alkali metal is sodium and wherein said sodium is present at less than about 100 ppm.

5. The process of claim 3 wherein said iron impurity is present at less than about 50 ppm.

6. The process of claim 1 wherein the catalyst contains about 0.1 to about 5 wt % hydrogenation catalyst metal.

7. The process of claim 1 wherein the hydrogenation catalyst metal comprises palladium.

8. The process of claim 1 wherein the aromatic carboxylic acid comprises terephthalic acid.

9. The process of claim 1 wherein the aromatic carboxylic acid comprises a crude aromatic carboxylic acid product obtained by liquid phase oxidation of a feed material comprising an aromatic compound with one or more substituents oxidizable to a carboxylic acid group and comprises aromatic carboxylic acid and at least one oxidation intermediate or by-product.

10. The process of claim 9 wherein the crude aromatic carboxylic acid product comprises terephthalic acid.

11. The process of claim 1 wherein the aromatic carbonyl compound comprises at least one of benzaldehyde, 2-, 3- and 4-carboxybenzaldehyde, 2,6-dicarboxyfluorenone and 2,6-dicarboxyanthroquinone.

12. A method of manufacturing a purified aromatic carboxylic acid product comprising:
contacting a feed material comprising an aromatic compound with about 0.3 to 0.5 wt % oxidizable substituents with oxygen in the presence of a heavy metal catalyst in a liquid reaction mixture under oxidation reaction conditions;
separating from the liquid reaction mixture a crude product comprising aromatic carboxylic acid and at least one oxidation intermediate or by-product; forming an aqueous solution comprising the crude product; and
contacting the aqueous solution with hydrogen in the presence of a catalyst in particulate form under hydrogenation reaction conditions at a temperature of about 200° to about 370° C., wherein the catalyst particles comprise a Group VIII hydrogenation catalyst metal dispersed homogeneously throughout a support comprising silicon carbide having a BET surface area of at least about 1 m²/g wherein the catalyst comprises less than about 100 ppmw iron and a total impurity level of less than 300 ppmw.

13. The method of claim 12 wherein said support further comprises titanium or a rare earth metal.

14. The method of claim 12 wherein the catalyst comprises less than about 50 ppmw iron.

15. The method of claim 12 wherein the oxidation intermediate or by-product comprises at least one carboxybenzaldehyde.

16. The method of claim 12 wherein the oxidation intermediate or by-product comprises 4-carboxybenzaldehyde.

17. The method of claim 12 wherein the catalyst metal comprises palladium.

18. A process for purification of an aromatic carboxylic acid product comprising terephthalic acid and about 0.3 to 0.5 wt % of at least one impurity comprising 4-carboxybenzaldehyde, hydroxymethyl benzoic acid, p-toluic acid, 2,6-dicarboxyfluorenone, 2,6-dicarboxyanthroquinone, 2,4′,5-tricarboxybiphenyl, 2,5-dicarboxyphenyl-4-carboxyphenyl methane, 3,4′- and 4,4′-dicarboxybiphenyl, and 2,6-dicarboxyfluorene comprising contacting an aqueous solution of the product with hydrogen in the presence of a catalyst in particulate form under hydrogenation reaction conditions at a temperature of about 200° to about 370° C., wherein the catalyst particles comprises a Group VIII hydrogenation catalyst metal dispersed homogeneously throughout a support comprising silicon carbide having a BET surface area of at least about 1 m²/g, the catalyst has an initial attrition loss according to ASTM D-4058 up to about 1.2 wt % and the silicon carbide comprises less than about 200 ppm impurities wherein at least 97 wt % of said impurity comprising 4-carboxybenzaldehyde, hydroxymethyl benzoic acid, p-toluic acid, 2,6-dicarboxyfluorenone, 2,6-dicarboxyanthroquinone, 2,4′,5-tricarboxybiphenyl, 2,5-dicarboxyphenyl-4-carboxyphenyl methane, 3,4′- and 4,4′-dicarboxybiphenyl, and 2,6-dicarboxyfluorene is removed.

19. The process of claim 18 wherein said impurities comprise iron and alkali metals.

20. The process of claim 18 wherein said catalyst comprises less than about 100 ppm iron and 100 ppm sodium.
